(19) United States
(12) Patent Application Publication
(10) Pub. No.: US 2018/0220895 A1
(43) Pub. Date: Aug. 9, 2018

(54) INFORMATION PROCESSING APPARATUS AND INFORMATION PROCESSING METHOD

(71) Applicant: CANON KABUSHIKI KAISHA, Tokyo (JP)

(72) Inventors: Fumitaro Masaki, Brookline, MA (US); Nobuhito Suehira, Tokyo (JP)

(21) Appl. No.: 15/879,558

(22) Filed: Jan. 25, 2018

(30) Foreign Application Priority Data
Feb. 3, 2017 (JP) 2017-018688

(51) Int. Cl. A61B 5/00

(52) U.S. Cl. CPC A61B 5/0073; A61B 5/0035; A61B 5/0095; A61B 5/0062; A61B 5/0048

(57) ABSTRACT

The present invention employs an information processing apparatus, comprising: an information processing unit configured to: acquire concentration information, which indicates a spatial distribution of concentration of a substance constituting an object and which originates from a photoacoustic wave generated by light irradiation of the object; and correct, based on the concentration in a specific position of the object in the concentration information, the concentration in a deep region of the object deeper than the specific position in the concentration information.

### BACKGROUND OF THE INVENTION

### Field of the Invention

The present invention relates to an information processing apparatus and an information processing method.

### Description of the Related Art

Photoacoustic tomography (PAT) has been receiving attention as a method for specifically imaging neovessels which are generated due to cancer. PAT is a technique to detect a photoacoustic wave, which is emitted from an object when the object is irradiated with pulsed light (near infrared), using an acoustic wave detector, and to image the detected photoacoustic wave.

In PAT, the initial sound pressure distribution P₀ of the photoacoustic wave, which is generated from a region of interest in the object, is given by Expression (1).

[Math. 1]

P₀=Γ·μₐ·Φ (1)

Γ here denotes a Grüneisen coefficient, which is determined by dividing the product of the volume expansion coefficient β and a square of the sound velocity c by a specific heat Cp. It is known that Γ is approximately constant if the object is determined. μₐ denotes an absorption coefficient in the region of interest, and ϕ denotes a light quantity in the region of interest (quantity of light irradiated to the region of interest, and is also called “optical fluence”).

The photoacoustic wave generated inside the object propagates inside the object, and is detected by an acoustic wave detector which is disposed on the surface of the object. Based on this detection result, the information processing apparatus acquires the initial sound pressure distribution P₀ using a reconstruction method, such as a back projection method.

As indicated in Expression (1), the distribution of the product of μₐ and ϕ, that is, the light energy density distribution, can be acquired by dividing the initial sound pressure distribution P₀ by the Grüneisen coefficient Γ. Further, the absorption coefficient distribution μₐ(r) is acquired by dividing the light energy density distribution by the light quantity distribution ϕ(r) inside the object. Furthermore, in the photoacoustic tomography, the spatial distribution of the concentration of a substance constituting the object can be calculated based on the acquired absorption coefficient distribution μₐ(r). As a method of acquiring the concentration of a substance constituting the object, Non-Patent Literature 1 discloses a method of calculating the oxygen saturation distribution using two absorption coefficient distributions acquired by using lights having two wavelengths.

Non Patent Literature 1: “Functional photoacoustic tomography for non-invasive imaging of cerebral blood oxygenation and blood volume in rat brains in vivo”, X. Wang, L. V. Wang, et al, Proc. of SPIE, Vol. 5697 (2005)

### SUMMARY OF THE INVENTION

In the spatial distribution of the concentration of a substance acquired by the photoacoustic tomography, however, acquisition accuracy may differ depending on a position inside the object.

With the foregoing in view, the present invention is realized. An object of the present invention is to provide a technique to accurately acquire the spatial distribution of a substance constituting the object in photoacoustic tomography.

The present invention provides an information processing apparatus, comprising:

an information processing unit configured to:

acquire concentration information, which indicates a spatial distribution of concentration of a substance constituting an object and which originates from a photoacoustic wave generated by light irradiation of the object; and

correct, based on the concentration in a specific position of the object in the concentration information, the concentration in a deep region of the object deeper than the specific position in the concentration information.

The present invention also provides an information processing method, comprising:

an information processing step of acquiring concentration information, which indicates a spatial distribution of concentration of a substance constituting an object, and which originates from a photoacoustic wave generated by light irradiation of the object; and

a correction step of, based on the concentration in a specific position of the object in the concentration information, correcting the concentration in a deep region of the object deeper than the specific position of the object in the concentration information.

According to the present invention, a technique to accurately acquire the spatial distribution of a substance constituting the object can be provided in photoacoustic tomography.

Further features of the present invention will become apparent from the following description of exemplary embodiments with reference to the attached drawings.

### BRIEF DESCRIPTION OF THE DRAWINGS

FIGS. 1A and 1B are diagrams depicting an object information acquiring apparatus according to Embodiment 1;

FIG. 2 is a flow chart depicting an object information acquiring method according to Embodiment 1;

FIG. 3 is a diagram depicting the oxygen saturation of an object according to Embodiment 1;

FIG. 4 is a diagram depicting a breast and blood vessels of the object according to Embodiment 1;

FIG. 5 is a diagram depicting the oxygen saturation of the object according to Embodiment 1;

FIG. 6 is a diagram depicting the breast and blood vessels of an object according to Embodiment 2;

FIG. 7 is a diagram depicting the oxygen saturation of the object according to Embodiment 2;

FIG. 8 is a diagram depicting the oxygen saturation of an object of which second component ratio is different;

FIG. 9 is a flow chart depicting an object information acquiring method according to Embodiment 3; and

FIG. 10 is a correction table that is used for the object information acquiring method according to Embodiment 3.

### DESCRIPTION OF THE EMBODIMENTS

Preferred embodiments of the present invention will be described below with reference to the drawings. The dimensions, materials, shapes and relative positions of the components described below should be changed appropriately depending on the configuration and various conditions of the apparatus to which the invention is applied. Therefore the scope of the present invention is not limited by the following description.

The present invention relates to a technique to detect an acoustic wave which propagates from an object, and generate and acquire characteristic information inside the object. This means that the present invention may be understood as: an object information acquiring apparatus or a control method thereof; an object information acquiring method; or a signal processing method. The present invention may also be understood as a program which causes an information processing apparatus equipped with such hardware resources as a CPU or memory to execute these methods, or a computer readable non-transitory storage medium storing this program.

The object information acquiring apparatus of the present invention includes a photoacoustic imaging apparatus using the photoacoustic effect, which is configured to receive an acoustic wave generated inside an object when the object is irradiated with light (electromagnetic wave), and acquire the characteristic information of the object as image data. In this case, the characteristic information is information on the characteristic value which is generated using a receive signal originating from the received photoacoustic wave and which corresponds to each of a plurality of positions inside the object.

The characteristic information acquired by the photoacoustic measurement refers to values reflecting the absorption amount and the absorption rate of the light energy. For example, the characteristic information includes a generation source of an acoustic wave which has been generated by irradiating light having a single wavelength, an initial sound pressure inside the object, and a light energy absorption density and an absorption coefficient which are derived from the initial sound pressure. Further, the concentration of a substance constituting a tissue can be acquired from the characteristic information acquired by lights having a plurality of mutually different wavelengths. As the substance concentration, an oxyhemoglobin concentration and a deoxyhemoglobin concentration can be determined. An oxygen saturation, that is determined from the oxyhemoglobin concentration and a deoxyhemoglobin concentration, can be a type of substance concentration. Further, as the substance concentration, a glucose concentration, a collagen concentration, a melanin concentration, and a volume percentage of fat, water or the like, may be determined. In the present invention, as the characteristic information, the oxygen saturation in the target inside the object, and the oxygen saturation inside the object will be described.

Based on the characteristic information at each position inside the object, a two-dimensional or three-dimensional characteristic information distribution is acquired. The distribution data can be generated as image data. The characteristic information may be determined as distribution information at each position inside the object, instead of as numeric data. In other words, such distribution information as initial sound pressure distribution, energy absorption density distribution, absorption coefficient distribution, substance concentration distribution, and oxygen saturation distribution may be determined.

The acoustic wave referred to in the present invention is typically an ultrasound wave, and includes elastic waves called a “sound wave” and an “acoustic wave”. An electric signal which has been converted from an acoustic wave by a transducer or the like is also called an “acoustic signal”. The ultrasound wave or acoustic wave that is referred to in this description is not intended to limit the wavelength of the elastic wave. An acoustic wave generated by the photoacoustic effect is called a “photoacoustic wave” or a “light-induced ultrasound wave”. An electric signal which originates from a photoacoustic wave is also called a “photoacoustic signal”. The distribution data is also called a “photoacoustic image data” or “reconstructed image data”.

In the following embodiment, a photoacoustic imaging apparatus, which acquires the distribution of light absorbers in an object by irradiating the object with pulsed light, and receiving and analyzing an acoustic wave from the object generated by the photoacoustic effect, will be described. This photoacoustic imaging apparatus is suitable for the diagnosis of vascular diseases and malignant cancers of humans and animals, or for the follow-up observation of chemotherapy. Examples of an object are a part of a living body (e.g. a breast and hand of a patient), an animal other than a human (e.g. mouse), an inanimate object and a phantom.

As a method of acquiring the concentration of a substance constituting an object, Non-Patent Literature 1 discloses a method of calculating the oxygen saturation distribution using two absorption coefficient distributions acquired using lights with two different wavelengths.

For example, it is assumed that the molar absorption coefficient of oxyhemoglobin is ε_{Hbo}(mm⁻¹ M⁻¹), and the molar absorption coefficient of deoxyhemoglobin ε_{Hb}(mm⁻¹ M⁻¹). Here the molar absorption coefficient is an absorption coefficient when there is 1 mol of hemoglobin per liter. The value of the molar absorption coefficient is uniquely determined by the wavelength.

It is also assumed that the molar concentration (M) of oxyhemoglobin is C_{Hbo}, and the molar concentration (M) of deoxyhemoglobin is C_{Hb}. In this case, the absorption coefficients μₐ of the blood at wavelengths λ₁ and λ₂ are given by the following Expression (2).

$$\begin{matrix}[ Math . 2 ] & \\ \{ \begin{matrix}{µ}_{a} ( {λ}_{1} ) = {ɛ}_{HbO} ( {λ}_{1} ) \cdot {C}_{HbO} + {ɛ}_{Hb} ( {λ }_{1} ) \cdot {C}_{Hb} \\ {µ}_{a} ( {λ}_{2} ) = {ɛ}_{HbO} ( {λ }_{2} ) \cdot {C}_{HbO} + {ɛ}_{Hb} ( {λ }_{2} ) \cdot {C}_{Hb}\end{matrix} & ( 2 )\end{matrix}$$

In other words, the absorption coefficient μₐ of the blood at each wavelength is given by the sum of: the product of the molar absorption coefficient ε_{Hbo} of the oxyhemoglobin, and the molar concentration C_{Hbo} of the oxyhemoglobin; and the product of the molar absorption coefficient ε_{Hb} of the deoxyhemoglobin and the molar concentration C_{Hb} of the deoxyhemoglobin.

Expression (2) can be transformed into Expression (3).

$$\begin{matrix}[ Math . 3 ] & \\ \{ \begin{matrix}{C}_{HbO} = \frac{{ɛ}_{Hb} ( {λ}_{1} ) \cdot {µ}_{a} ( {λ}_{2} ) - {ɛ}_{Hb} ( {λ}_{2} ) \cdot {µ}_{a} ( {λ}_{1} )}{{ɛ}_{Hb} ( {λ}_{1} ) \cdot {ɛ}_{HbO} ( {λ}_{2} ) - {ɛ}_{HbO} ( {λ}_{1} ) \cdot {ɛ}_{Hb} ( {λ}_{2} )} \\ {C}_{Hb} = \frac{{ɛ}_{Hb} ( {λ}_{1} ) \cdot {µ}_{a} ( {λ}_{2} ) - {ɛ}_{HbO} ( {λ}_{2} ) \cdot {µ}_{a} ( {λ}_{1} )}{{ɛ}_{HbO} ( {λ}_{1} ) \cdot {ɛ}_{Hb} ( {λ}_{2} ) - {ɛ}_{Hb} ( {λ}_{1} ) \cdot {ɛ}_{Hb} ( {λ}_{2} )}\end{matrix} & ( 3 )\end{matrix}$$

The oxygen saturation degree StO₂, which is a ratio of the oxyhemoglobin to the total hemoglobin, can be given by the following Expression (4).

$$\begin{matrix}[ Math . 4 ] & \\ {StO}_{2} = \frac{{C}_{HbO}}{{C}_{HbO} + {C}_{Hb}} = \frac{- {µ}_{a} ( {λ}_{1} ) {ɛ}_{Hb} ( {λ}_{2} ) + {µ}_{a} ( {λ}_{2} ) {ɛ}_{Hb} ( {λ}_{1} )}{\begin{matrix}- {µ}_{a} ( {λ}_{1} ) \{ {ɛ}_{Hb} ( {λ}_{2} ) - {ɛ}_{HbO} ( {λ}_{2} ) \} + \\ {µ}_{a} ( {λ}_{2} ) \{ {ɛ}_{Hb} ( {λ}_{1} ) - {ɛ}_{HbO} ( {λ}_{1} ) \}\end{matrix}} & ( 4 )\end{matrix}$$

In other words, if the absorption coefficients μₐ at the wavelengths λ1 and λ2 are known, the oxygen saturation can be calculated by Expression (4), since the other values of Expression (4) are known.

In the spatial distribution of the oxygen saturation that is acquired by the method of Non-Patent Literature 1, the acquisition accuracy may be different depending on the position inside the object.

### Embodiment 1

An embodiment of the object information acquiring apparatus according to the present invention will be described in detail with reference to the drawings.

Apparatus Configuration

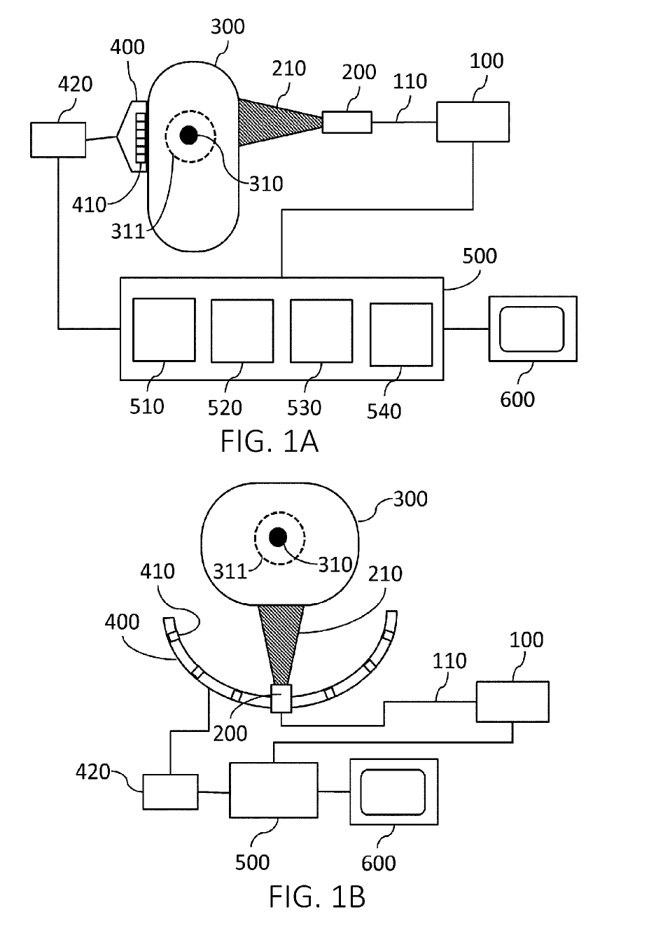

FIG. 1A is a block diagram depicting a general configuration of an object information acquiring apparatus of Embodiment 1. The apparatus includes a light source 100, an irradiation optical system 200, an acoustic wave detector 400, a signal acquiring apparatus 420, an information processing apparatus 500, and a display device 600.

Light Source 100

The light source 100 is an apparatus that emits light 110 in order to generate a photoacoustic wave from the surface of the object and inside of the object by the photoacoustic effect. To calculate the oxygen saturation, the light source 100 is constructed such that lights having a plurality of wavelengths, including at least a light having a first wavelength λ₁, and a light having a second wavelength λ₂ which is different from the first wavelength can be generated. In some cases, an acoustic wave that originates from the light having the first wavelength may be called a “first acoustic wave”, and an acoustic wave that originates from the light having the second wavelength may be called a “second acoustic wave”. Further, a photoacoustic signal that originates from the first acoustic wave may be called a “first signal”, and a photoacoustic signal which originates from the second acoustic wave may be called a “second signal”.

It is preferable that the light source 100 can generates pulsed light, of which pulse width is 5 to 50 ns. In order to calculate the oxygen saturation distribution in a region at a several tens mm depth, the light source 100 is preferably a laser device having high output (e.g. at least several mJ/pulse). For the laser, a solid-state laser, a gas laser, a dye laser, a semiconductor laser or the like can be used. In particular, a Ti:sa laser excites by an Nd:YAG, or an alexandrite laser is preferable, because the output is high, and the wavelength can be continuously changed.

To make the wavelength variable, the light source 100 may be constituted by single wavelength lasers having different wavelengths. In this case, the light source 100 includes a first light source which generates a light having a first wavelength, and a second light source which generates a light having a second wavelength. The light source 100 may be constituted by a light-emitting diode and a flash lamp or the like, instead of a laser device.

Irradiation Optical System 200

The irradiation optical system 200 is an optical system which irradiates the object 300 with the light 110 generated by the light source 100 as irradiation light 210. The irradiation optical system 200 is constituted by optical members for guiding the light 110, and generating a desired irradiation profile. The optical members are, for example, a mirror that reflects light, a half-mirror that branches the reference light and the irradiated light, a lens that changes the shape of the light by condensing or expanding, a diffusion plate that expands the light, and a fiber bundle. It is preferable that the irradiation light 210 can be expanded to a certain area. A diffusion plate, a fly eye lens and the like may be used to smooth the light intensity distribution of the irradiation light 210.

It is preferable that the irradiation optical system 200 can irradiate the object 300 with the irradiation light 210, of which optical pattern is the same at each wavelength. If the irradiation optical system 200 is a fiber bundle, the optical pattern at each wavelength may be made to be the same by making the arrangement of the emitting ends of the fiber bundle random with respect to the entry ends of the fiber bundle.

It is preferable that the irradiation region of the irradiation light 210 can move on the surface of the object 300. If the irradiation region moves on the surface of the object 300, the light can be irradiated and superimposed on the same region, hence the light intensity on the surface of the object becomes uniform. As a result, it is possible to obtain the same effect as the case of irradiating the object with lights having the same light intensity distribution at two wavelengths. The method of moving the irradiation region on the object 300 is, for example, a method of using a movable mirror, or a method of mechanically moving the irradiation optical system 200 itself.

In FIG. 1A, the irradiation light 210 may be irradiated from the acoustic wave detector 400 side, or from the opposite side of the acoustic wave detector 400. The irradiation light 210 may be irradiated from both sides of the object 300.

Object 300

The object 300 is not a composing element of the object information acquiring apparatus, but will be described here nonetheless. In the case of using the object information acquiring apparatus for the diagnosis of vascular diseases and malignant cancers of humans and animals, or for the follow-up observation of chemotherapy, the object is assumed to be a living body (e.g. a breast, head, neck, abdomen of a human or animal). The light absorber is an area which has a relatively high light absorption coefficient inside the object. The light absorber is, for example, oxyhemoglobin or deoxyhemoglobin, or a blood vessel which contains a high amount of oxyhemoglobin or deoxyhemoglobin, or a malignant cancer which includes many neovessels.

It is preferable that an acoustic matching material is disposed between the object 300 and the acoustic wave detector 400, so that the acoustic impedance there between is matched and the photoacoustic wave is propagated efficiently. For the acoustic matching material, water, oil, gel or the like is suitable.

It is preferable to dispose a holding member (not illustrated) to hold the object 300. By using the holding member, body motion during the measurement, which drops image quality, can be suppressed. Further, the holding member stabilizes the shape of the object 300, whereby the irradiation range and the irradiation position of the light irradiated to the surface of the object can be more easily determined. The holding member is preferably constituted by a material which has a certain degree of strength, and does not deform, and allows the acoustic wave propagated from the object 300 and the irradiation light 210 to transmit through. For example, polycarbonate, polyethylene terephthalate, acryl or the like is suitable. If the object information acquired apparatus includes the holding member, the above mentioned acoustic matching material is disposed between the holding member and the object 300, and between the holding member and the acoustic wave detector 400.

Acoustic Wave Detector 400

The acoustic wave detector 400 is constituted by an element 410 and a support that supports the element. When the irradiation light 210, which is irradiated to the object 300, propagates inside the object, and is absorbed by a light absorber 310, the element 410 detects a photoacoustic wave 311 generated from the light absorber 310, and converts the photoacoustic wave 311 into an analog electric signal. For the element 410 used for the acoustic wave detector 400, for example, a transducer using piezoelectric phenomena, a transducer using the resonance of light, and a transducer using the change in capacitance, can be used. In the acoustic wave detector 400, the elements 410 may be arrayed as illustrated, or a single element may be used.

It is preferable that the relative position of the acoustic wave detector 400, with respect to the object 300, is changeable. If the relative positions of the acoustic wave detector 400 and the object 300 can be changed, a wide range of the object 300 can be imaged. For the position changing mechanism, a device having a power mechanism and a positioning mechanism, such as an XY stage, is preferable. The acoustic wave detector 400 may be a handheld type probe which has a gripper. In the case of a handheld type probe, the light-emitting end of the irradiation optical system 200 is preferably inside the probe.

A signal acquiring apparatus 420 performs amplification processing and digital conversion processing on the analog electric signal (detection signal) outputted from the acoustic wave detector 400. The signal acquiring apparatus 420 is constituted by an amplifier device, and A/D converting circuit and the like. In the present description, the “detection signal” includes both the analog signal outputted from the acoustic wave detector 400, and the digital signal converted from the analog signal.

Information Processing Apparatus 500

The information processing apparatus 500 is an apparatus for acquiring object information based on the detection signal outputted from the signal acquiring apparatus 420. The information processing apparatus 500 acquires the optical characteristic value distribution inside the object by reconstructing the image based on the detection signal. For the information processing apparatus 500, a PC or workstation, which includes such arithmetic resources as a CPU and memory, and operates based on programmed commands and input information from the user, is preferable. Each block included in the information processing apparatus 500 may be constituted by an independent PC, or may be constituted by a program module which operations in the same PC. The information processing apparatus 500 corresponds to the information processing unit of the present invention. The information processing apparatus 500 may function as a correction unit of the present invention.

For the image reconstruction algorithm, a known processing which is normally used in tomography techniques can be used. For example, a delay and sum method, a reverse projection method in the time domain or Fourier domain or the like can be used. In the case when the capability of the information processing apparatus 500 is high, or when sufficient time can be spent for image reconstruction (e.g. in the case of performing image reconstruction at a timing that is different from the photoacoustic wave acquisition), such an image reconstruction method as an inverse problem analysis method based on repeat processing can be used. By using an acoustic wave detector including an acoustic lens or the like, the information processing apparatus 500 can generate the initial sound pressure distribution inside the object without performing the image reconstruction. In the case when a plurality of detection signals are acquired from the acoustic wave detector 400, it is preferable that the information processing apparatus 500 can simultaneously process the plurality of signals. Thereby the image forming time can be reduced.

The functional blocks of the information processing apparatus 500 will be described next. As mentioned above, these blocks need not be physically separated. Each functional block may be constructed as a program module. The information processing apparatus 500 may be constituted by a combination of a plurality of PCs. Further, the information processing apparatus 500 may process the detection signal, which is outputted from the signal acquiring apparatus 420 and stored in a storage device, using a cloud. The information processing apparatus 500 preferably includes an input device (e.g. mouse, keyboard, touch panel) as a user interface which receives input from the user.

An apparatus control unit 510 controls the operation content and operating timing of each block of the object information acquiring apparatus, in accordance with the instructions of the program which is generated and stored in the storage device in advance, and the instructions from the user via the input device. A reconstruction processing unit 520 performs image reconstruction processing using the above mentioned method, for the detection signal acquired by the photoacoustic measurement, in which the object is irradiated with light and the photoacoustic wave is received. A reconstruction processing unit 520 also has a function to determine the absorption coefficient distribution from the reconstructed initial sound pressure distribution.

A blood vessel extracting unit 530 extracts blood vessels from the reconstructed image (e.g. initial sound pressure distribution image, absorption coefficient distribution image, light energy absorption density distribution image) outputted from the reconstruction processing unit 520. The method of extracting the blood vessels is arbitrary, such as: a method of regarding a region, in which the values of the optical characteristic values are higher than a predetermined threshold, as a blood vessel; a pattern matching method; and an image recognition method. An oxygen saturation acquiring unit 540 has a function to determine the oxygen saturation distribution from an absorption coefficient distribution with a plurality of wavelengths. The oxygen saturation acquiring unit 540 also has functions to determine a later mentioned simplified oxygen saturation distribution (first oxygen saturation distribution), to determine a high precision oxygen saturation distribution (third oxygen saturation distribution), and to determine a final oxygen saturation distribution for display (second oxygen saturation distribution).

Display Device 600

The display device 600 displays object information that is outputted from the information processing apparatus 500. For the display device 600, a liquid crystal display, a plasma display, an organic EL display or the like can be used. Before displaying the object information, image processing such as image quality correction and brightness adjustment may be performed first by the display device 600 or by the information processing apparatus 500. The display device 600 may be integrated with the object information acquiring apparatus, or may be separate from the object information acquiring apparatus.

Modification

FIG. 1B depicts another form of the object information acquiring apparatus to which the present invention can be applied. A block the same as FIG. 1A is denoted by the same reference sign. The internal configuration of the information processing apparatus 500 is omitted. An acoustic wave detector 400 in FIG. 1B is constituted by a bowl-shaped support and a plurality of elements 410 which are disposed on the inner peripheral surface of the support. On the base of the bowl-shaped support, the irradiation optical system 200 is disposed. Since this configuration allows receiving the photoacoustic waves, generated by the object 300, from many directions, the image quality of the reconstructed image improves. It is also preferable to dispose a scanning mechanism to move the bowl-shaped support, so that a wide range can be imaged.

Processing Flow of Object Information Acquiring Method

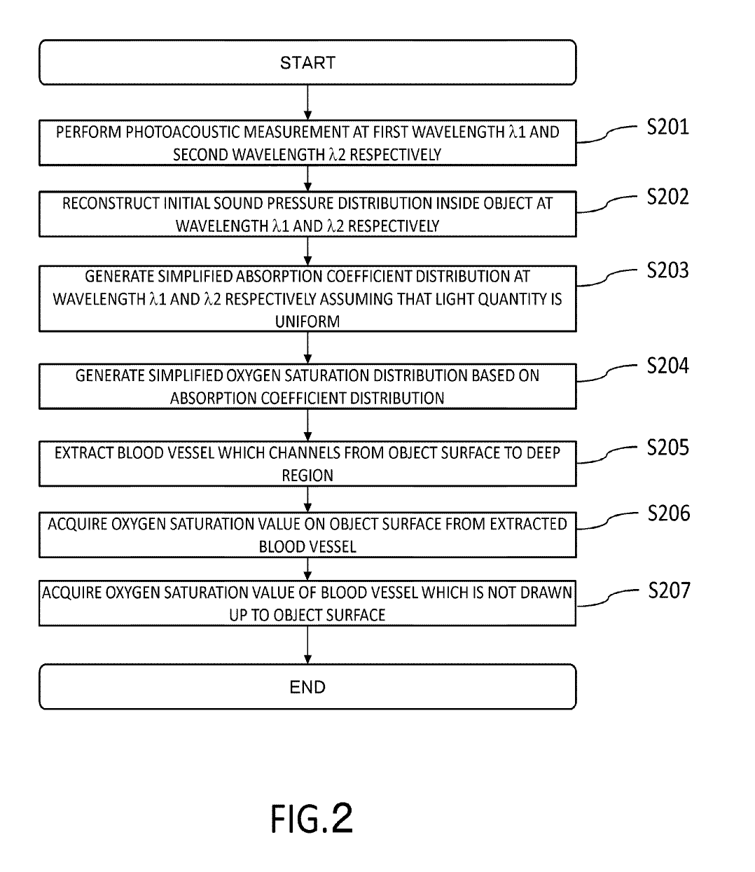

The processing flow of this embodiment will be described next with reference to FIG. 2.

In step S201, the photoacoustic measurement is performed at a first wavelength λ1 and a second wavelength λ2 respectively, and a detection signal at each wavelength is acquired respectively. In other words, the light 110 having the first wavelength emitted from the light source 100 is irradiated to the object 300 as the irradiation light 210 via the irradiation optical system 200. The light absorber 310 inside the object 300 absorbs the irradiation light 210, and generates the photoacoustic wave 311.

The acoustic wave detector 400 detects the photoacoustic wave, and converts the photoacoustic wave into an electric signal as a first signal. In the same manner, the acoustic wave detector 400 detects the photoacoustic wave 311 which is generated when the object 300 is irradiated with the irradiation light 210 having the second wavelength, and converts the photoacoustic wave into an electric signal as a second signal. The detected signals corresponding to the first and second wavelengths respectively, converted into digital signals by the signal acquiring apparatus 420, are stored in the storage device.

In step S202, the reconstruction processing unit 520 of the information processing apparatus 500 reconstructs the initial sound pressure distribution inside the object at wavelengths λ1 and λ2 respectively. In other words, based on the first signal and the second signal respectively, the information processing apparatus 500 acquires a first initial sound pressure distribution which originates from the first wavelength, and a second initial sound pressure distribution which originates from the second wavelength.

In step S203, the reconstruction processing unit 520 of the information processing apparatus 500 generates a simple absorption coefficient distribution respectively with wavelengths λ1 and λ2, assuming that light quantity is uniform inside the object. In other words, the distribution of μₐ is calculated using Expression (1) without considering the influence of absorption and scattering. The quantity of light which is irradiated to the surface of the object in this case may be called a “first light quantity”, and the light quantity distribution which is specified based on the first light quantity assuming that the light quantity is uniform inside the object may be called a “first light quantity distribution”. On the other hand, the high precision light quantity distribution, which is determined using the first light quantity and the effective attenuation coefficient inside the object may be called a “second light quantity distribution”.

In step S204, the oxygen saturation acquiring unit 540 of the information processing apparatus 500 generates the oxygen saturation distribution using Expression (4) based on the absorption coefficient distribution at each wavelength which has been determined in the simplified process in step S203. The oxygen saturation distribution acquired here is a simplified oxygen saturation distribution which has been calculated assuming that the light quantity is uniform. This is also called a “first oxygen saturation distribution”.

Relationship of Light Quantity and Oxygen Saturation Degree Value

The light behavior of the light, which is irradiated to the surface of the object, inside the object, will be described. The light quantity when the light propagates inside a scatterer can be given by the following Expression (5) if it is assumed that the model is one-dimensional.

ϕ(d)=ϕ₀·exp(−μeff·d) (5)

ϕ₀ denotes a quantity of incident light that enters the scatterer, μ_{eff} denotes the effective attenuation coefficient of the scatterer, and d denotes a distance that the light traveled. Here conversion of the model in Expression (5) into a three-dimensional model is considered. If the surface of the object is regarded as a plane spreading in the xy directions, the z direction indicates the depth direction. When the light is irradiated approximately vertical to the object as shown in FIGS. 1A and 1B, the z direction approximately matches the light irradiating direction, and indicates the distance from the light irradiation surface. In this case, to accurately calculate the light quantity distribution ϕ (x, y, z) inside the object, the Monte Carlo method or the finite element method must be used, as mentioned in Non-Patent Literature 1. However, the light quantity distribution ϕ (x, y, 0) on the surface of the object (that is, z=0) can easily be measured using a photodiode, a CCD camera or the like. Further, the light quantity distribution on the surface can be accurately calculated from the result of ray tracing executed by the irradiation optical system 200.

The procedure to derive the absorption coefficient distribution μₐ (x, y, 0) on the surface of the object using Expression (1) is considered. The initial sound pressure distribution P₀ is acquired by the reconstruction method, such as back projection. The Grüneisen coefficient Γ becomes an approximately constant value if the object is determined. The light quantity distribution ϕ (x, y, 0) on the surface of the object can be determined by measurement or calculation as mentioned above. Since all information of the initial sound pressure distribution P₀, the Grüneisen coefficient Γ and the light quantity distribution ϕ (x, y, 0) on the surface of the object can be obtained, the absorption coefficient distribution μₐ (x, y, 0) on the surface of the object can be accurately determined.

In a range where the depth z is sufficient shallow, that is, in a region near the surface of the object, there is no problem even if a simplified absorption coefficient distribution μₐ (x, y, z) is derived using the light quantity distribution ϕ (x, y, 0) where z=0. However as the region becomes deeper in the object, the amount of deviation between the absorption coefficient value, when the light quantity distribution is accurately estimated, and the absorption coefficient value, which is determined in a simplified process, increases. For example, when the effective attenuation coefficient is 0.1 [mm⁻¹], the amount of deviation there between is about 5%, which is sufficiently small, if the region is in a 0.5 mm range from the surface of the skin. Further, the oxygen saturation of a blood vessel is calculated from the absorption coefficient distributions acquired at a plurality of wavelengths, as shown in Expression (3), hence if the amount of deviation between the simplified value of the absorption coefficient distribution and the accurately estimated value increases, the amount of deviation of the calculated oxygen saturation value from the actual value increases as well.

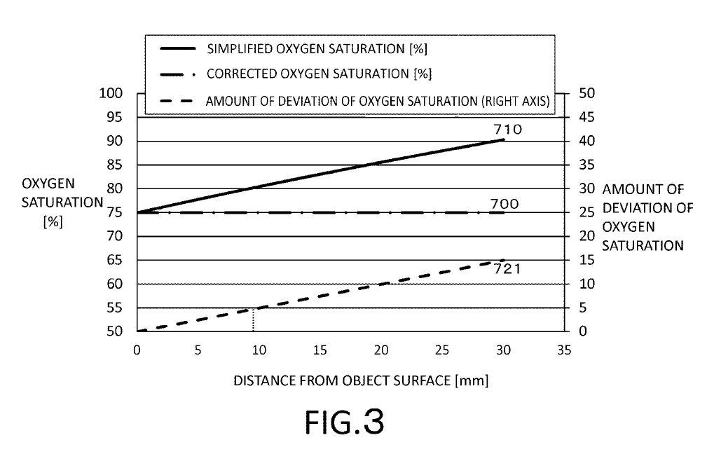

A region near the surface of the object is a range of which depth from the surface of the object is preferably within 0.5 mm, as mentioned above. In this case, the region, in which the propagation distance of the light from the position, where the light entered on the surface of the object, is within 0.5 mm, is the region near the surface of the object. However this depth is not limited to 0.5 mm. The “region near the surface of the object” may be defined with reference FIG. 3 based on the allowable amount of deviation of the oxygen saturation value which is set. For example, if the allowable amount of deviation is maximum 5%, the region near the surface of the object is down to a position of which depth from the surface of the object is about 9 mm (position of which light propagation distance from the incident position of the light is about 9 mm) indicated by the reference sign A in FIG. 3.

FIG. 3 shows: a high precision oxygen saturation 700 which is determined considering the light quantity distribution in the depth direction; an oxygen saturation 710 which is determined assuming that there is not light quantity distribution in the depth direction, in other words, which is determined in a simplified process by applying the light quantity distribution ϕ (x, y, 0) on the surface of the object for all the depths (hereafter called a “simplified oxygen saturation”); and a difference 721 between the high precision oxygen saturation 700 and the simplified oxygen saturation 710 (hereafter called an “amount of deviation of the oxygen saturation”). Here the optical constant is determined assuming that the object is a human breast, the wavelengths are 755 nm and 797 nm, and the calculation target is a vein. As shown in FIG. 3, the amount of deviation increases as the location is deeper in the breast. On the other hand, the amount of deviation is 0 on the surface, and the difference between the high precision oxygen saturation 700 and the simplified oxygen saturation 710 is sufficiently small in a region near the surface.

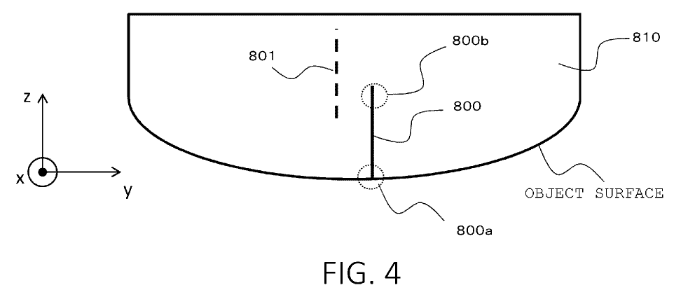

FIG. 4 is a schematic diagram depicting the object 810 (breast of the examinee) viewed from the cephalocaudal direction. The reference signs 800 and 801 are different blood vessels. For the blood vessel 800, the oxygen saturation value determined in the simplified process approximately matches the oxygen saturation value determined by accurately calculating the light quantity (amount of deviation being 0) in a region near to the surface of the object, which is the breast (corresponding to the reference sign 800a). However, as the value in the depth direction (z direction) increases and the region becomes deeper in the object (e.g. position corresponding to the reference sign 800b), the amount of deviation increases. The deep region of the object is a position of which distance from the surface of the object on the normal line that is on the surface and intersects a specific position of the object for which substance concentration is determined, is longer than that of the specific position.

However, since the metabolism of oxygen is generally performed in a capillary region, the oxygen saturation of artery and vein vessels in a region near the surface of the breast, and the oxygen saturation of the blood vessels in the deep part of the breast, are approximately the same. Therefore for the blood vessel 800, which channels to the region near the surface of the breast, the oxygen saturation value in a region near the surface of the breast (that is, the position corresponding to the reference sign 800a) is the accurate value. This means that the accurate oxygen saturation values can be indicated for the entire blood vessel by applying the oxygen saturation in a region near the surface to all the depths. A blood vessel of which at least a part is located in a region near the surface of the object, such as the blood vessel 800, is called a “first blood vessel”.

Referring back to the flow chart, the above description corresponds to steps S205 to S206. In other words, in step S205, the blood vessel extracting unit 530 of the information processing apparatus 500 extracts the blood vessel 800 which channels from the surface of the object to a deep region of the object, using such a method as threshold processing, pattern matching, and image recognition, or by receiving an instruction from the user via an input device (e.g. mouse, touch panel). The image that is used for extracting the blood vessel may be an image originating either from the light having the first wavelength or the light having the second wavelength, or may be either the initial sound pressure distribution image or the absorption coefficient distribution image.

In step S206, the oxygen saturation acquiring unit 540 of the information processing apparatus 500 acquires the oxygen saturation value of the extracted blood vessel 800 in the region near the surface of the object from the simplified oxygen saturation distribution, and applies this value to the entire blood vessel 800. This value is also called a “surface oxygen saturation value”, and corresponds to the first concentration information in this embodiment.

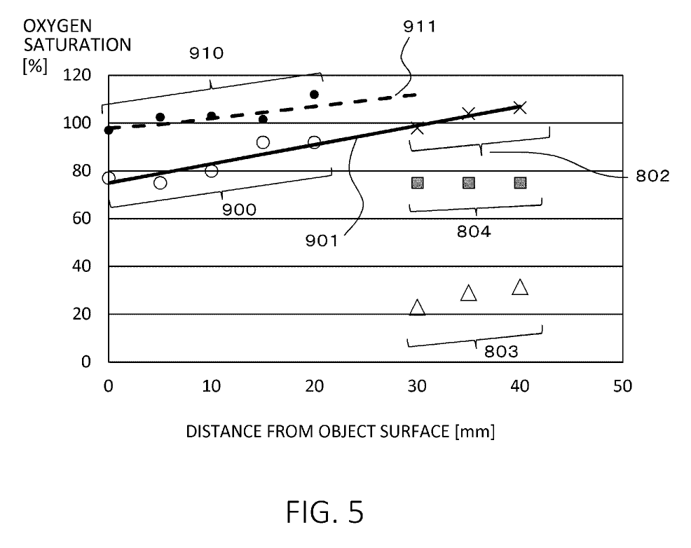

Then in step S207, an oxygen saturation value of a blood vessel, of which only a part is drawn without reaching the surface of the object, is acquired in the photoacoustic image, such as the blood vessel 801 in FIG. 4. A blood vessel which has no portion in the region near the surface of the object is also called a “second blood vessel”. To acquire the oxygen saturation value for such a blood vessel, it is necessary to plot in advance the dependency of the simplified oxygen saturation on the depth direction for at least one blood vessel which channels to a region near the surface. In the case of the example in FIG. 5, the dependency of the simplified oxygen saturation on the depth direction is plotted for a plurality of blood vessels which channel to a region near the surface, as in the case of the reference signs 900 and 910. The lines (901 and 911) are lines generated by extrapolating the acquired plotted points for each blood vessel.

Then the dependency 802 of the simplified oxygen saturation of the blood vessel 801 in the depth direction is plotted. Here the relationship between the oxygen saturation value and the distance from the surface of the object (light propagation distance) for the blood vessel 800, of which part is located in a region near the surface of the object, in the oxygen saturation distribution, is called a “first relationship”. The relationship between the oxygen saturation value and the distance from the surface of the object (light propagation distance) for the blood vessel 801, of which part is not located in a region near the surface of the object, on the other hand, is called a “second relationship”.

Then, the plotted dependency 802 in the depth direction is compared with the lines 901 and 911, and the line 901 is selected as a line which best expresses the dependency of the oxygen saturation of the blood vessel 801 in the depth direction. In other words, the oxygen saturation acquiring unit 540 acquires information to determine the oxygen saturation value of the blood vessel 801 by comparing the first relationship and the second relationship for the slope of the line and the like.

For the blood vessel corresponding to the line 901 which is drawn up to the portion in the region near the surface of the object, the simplified oxygen saturation in the region near the surface can be acquired. In other words, the oxygen saturation value at the distance z=0 [mm] from the surface on the plotted line indicated by the reference sign 900 can be acquired as the oxygen saturation value of the blood vessel 801. Alternatively, the amount of deviation (803) between the oxygen saturation on the surface of the vessel corresponding to the reference sign 900, and the simplified oxygen saturation of the blood vessel 801, may be calculated so that the oxygen saturation indicated by the reference sign 801 is corrected by subtracting this amount of deviation, which is the correction amount, from the dependency indicated by the reference sign 802. The accurate oxygen saturation values of the blood vessel 801 are the values indicated by the reference sign 804 in FIG. 5.

In step S207, the oxygen saturation values can be acquired not only for the blood vessels which are drawn up to the region near the surface of the object, but also for the blood vessels which are not drawn up to the surface of the object. The information processing apparatus 500 may output the image data, corresponding to the oxygen saturation distribution in the blood vessel region acquired by the processing up to S207, to the display device 600, and display this image data as the oxygen saturation distribution image inside the object. In this case, a display method which makes it easier to distinguish between the blood vessel regions and other regions, such as decreasing the brightness of portions other than the blood vessel regions, may be used. The oxygen saturation values of the entire blood vessel, determined based on the surface oxygen saturation value, is called a “second oxygen saturation value”, and the distribution of the second oxygen saturation values is called a “second oxygen saturation distribution”.

By performing the above steps, the oxygen saturation distribution in a deep region of the object can be accurately acquired merely by acquiring the light quantity distribution on the surface of the object, without calculating the light quantity distribution using a complicated calculation method, such as the Monte Carlo method and the finite element method.

In Embodiment 1, the oxygen saturation is corrected for all the depths. However the correction target region may be limited to only a deep part of the object, since the simplified oxygen saturation value, which has been determined assuming that the light quantity is uniform, can be used for the region near the surface of the object with relatively high reliability. The boundary of the depth (or the light propagation distance) from which the oxygen saturation is corrected can be appropriately determined depending on the accuracy of the information required by the user, and the accuracy of the simplified oxygen saturation value.

### Embodiment 2

In Embodiment 2, an example of applying the present invention considering the type of the blood vessel will be described. A composing element or a method the same as Embodiment 1 is denoted with the same reference sign, for which detailed description is omitted. The processing described below is executed by each functional block of the information processing apparatus 500 based on such an instruction as a program.

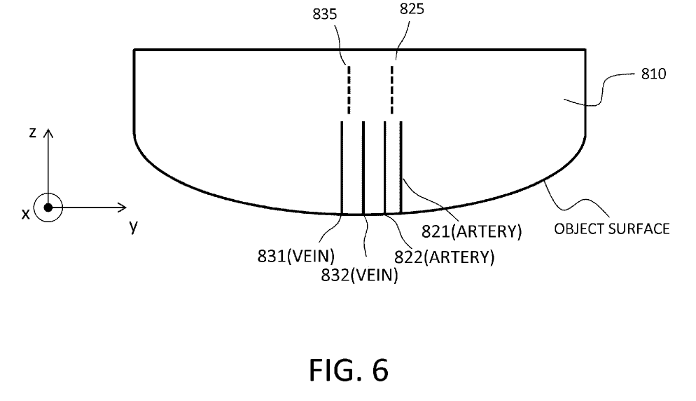

FIG. 6 is a PAT image of Embodiment 2. In FIG. 6, a plurality of blood vessels (artery 821, artery 822, vein 831, vein 832), are channeling to the region near the surface of the breast (object). The blood vessels are roughly classified into arteries and veins. In FIG. 6, the oxygen saturation of the arteries 821 and 822 are relatively high. The oxygen saturation of the veins 831 and 832, on the other hand, are relatively low.

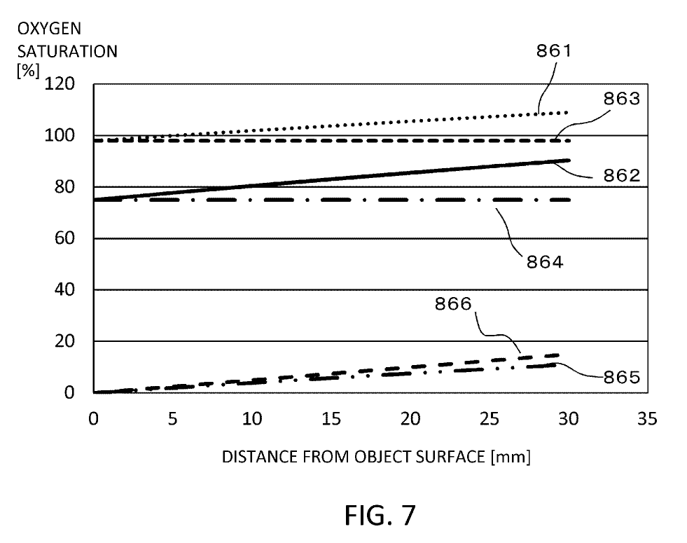

FIG. 7 is a graph depicting the values related to the oxygen saturation which have been calculated for the arteries and veins respectively, under the same calculation conditions as the case of FIG. 3. The values related to the oxygen saturation include the following:

(a) a high precision oxygen saturation value, which is calculated after accurately determining the light quantity distribution in the depth direction,
(b) a simplified oxygen saturation value, which is calculated assuming that the light quantity distribution is uniform, and
(c) the amount of the deviation between (a) and (b).

In concrete terms, (a) corresponds to the reference sign 861 (artery) and 862 (vein). (b) corresponds to the reference sign 863 (artery) and 864 (vein). (c) corresponds to the reference sign 865 (artery) and the reference sign 866 (vein). The amount of deviation of the oxygen saturation is different between the artery and the vein, primarily because the optical constants are different between the artery and the vein.

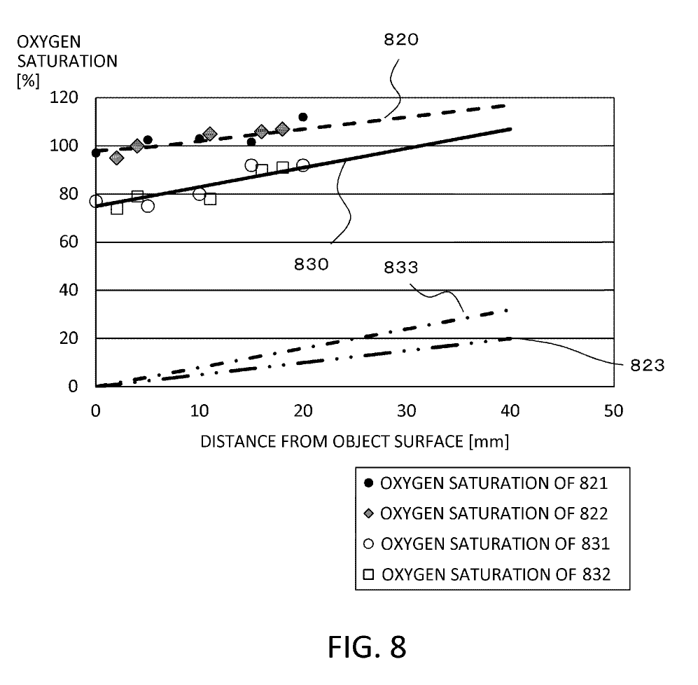

Here, using the same method as Embodiment 1, the dependency of the simplified oxygen saturation on the depth direction is plotted for the blood vessels, which channel to the region near the surface of the breast, as shown in FIG. 8. Then the plotting results can be roughly classified into two groups, although dispersion due to calculation or measurement errors exists. In other words, in FIG. 8, a line 820, which approximates the plotting result of the artery 821 and the artery 822, and a line 830, which approximates the plotting result of the vein 831 and the vein 832, can be drawn. Here the lines 820 and 830 are determined by the least square method. Therefore the line 820 is a line expressing the simplified oxygen saturation of the artery, and the line 830 is a line expressing the simplified oxygen saturation of the vein.

Then the differences (823, 833) between these lines and the oxygen saturation on the surface of the breast are determined respectively. Here the reference sign 823 is a line which indicates a correction amount for the artery, and the reference sign 833 is a line which indicates a correction amount for the vein. By subtracting this correction amount from the simplified oxygen saturation, an accurate oxygen saturation is acquired.

A plurality of blood vessels (825, 835 in FIG. 6), of which only a part of the blood vessel is extracted, will be described next. The information processing apparatus 500 compares the simplified oxygen saturation between blood vessels at the same depth. Normally the oxygen saturation of the artery and the vein are about 98% and about 75% respectively, indicating a sufficient difference, hence the blood vessels can be classified into two groups (arteries and veins) by comparing the simplified oxygen saturation determined at a same depth. In other words, a blood vessel of which the simplified oxygen saturation is high is an artery, and a blood vessel of which a simply determined oxygen saturation is low is a vein.

By subtracting the correction amounts 823 and 833 from the simplified oxygen saturation for the artery and the vein respectively, accurate oxygen saturation can be acquired. If the simplified oxygen saturation has no minor differences between the blood vessels, then these blood vessels may be the same type. In this case, the value used for correction may be determined by estimating whether the blood vessels are arteries or veins based on the simplified oxygen saturation values.

According to the method of Embodiment 2, the arteries and the veins are classified, and correction suitable for each type of blood vessels can be performed, hence a more accurate oxygen saturation distribution can be acquired.

### Embodiment 3

In Embodiment 1 and Embodiment 2, the correction amount of the oxygen saturation value is determined based on the simplified oxygen saturation acquired for each examinee. In Embodiment 3, based on the attributes of the examinee, the amount of deviation of the simplified oxygen saturation value from the high precision oxygen saturation is determined, and correction is performed. The correction information (correction table or correction formulae) corresponds to the first concentration information in Embodiment 3. The correction information is generated based on: a first oxygen saturation distribution determined from the first light quantity distribution, which is tentative and uniform, and is created based on the first light quantity on the surface of the object; and a third oxygen saturation distribution determined from the second light quantity distribution which is highly accurate. By applying the correction information selected in accordance with the attributes of the examinee to the first oxygen saturation distribution of the examinee, the second oxygen saturation distribution used for display is generated.

a: Correction Table Creation Process

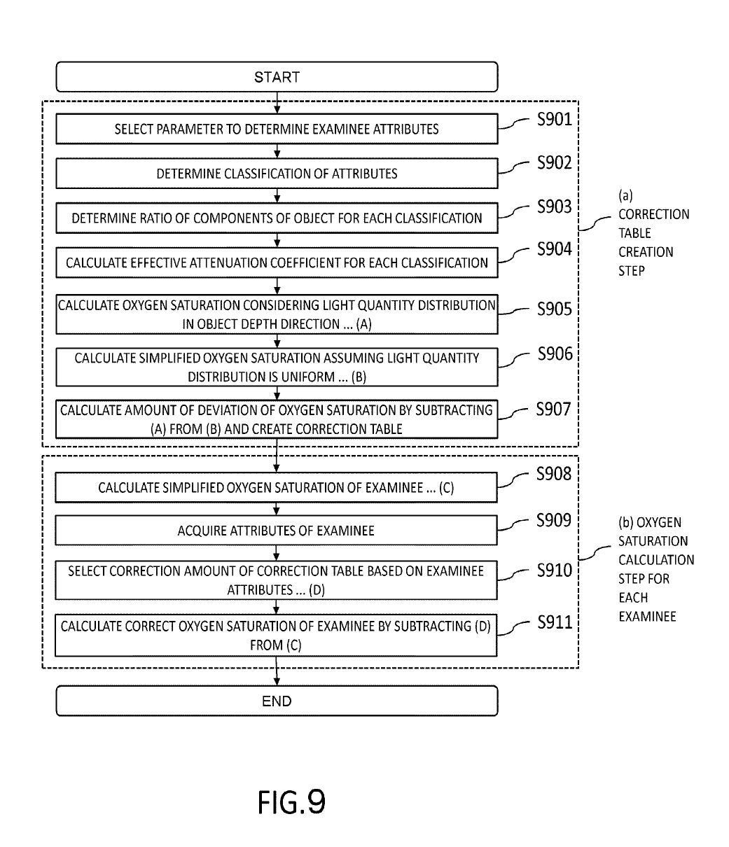

FIG. 9 is a flow chart depicting the correction table creating step and the oxygen saturation deriving step for each examinee. Step (a) is assumed to be performed before the actual photoacoustic measurement, but may be performed when a certain photoacoustic measurement is performed. The correction table created in step (a) is stored in a storage device (not illustrated).

In step S901, parameters to determine the attributes of the examinee are selected. Here age and body mass index (BMI) are used. The parameters to determine the attributes of the examinee are not limited to these. For example, height, weight, breast size, race, medical history and the like may be used as parameters to determine the attributes of the examinee.

As Expression (5) shows, the light quantity distribution greatly depends on the effective attenuation coefficient μ_{eff} of the scatterer. It is known that the effective attenuation coefficient depends on the ratio of components constituting the breast, such as the ratio of blood, fat, water and the like. The density of the mammary gland, and the amount of melanin on the surface of the skin also influences the effective attenuation coefficient. The ratio of the components constituting the breast can be estimated by the attributes of the examinee. For example, it is generally known that the density of the mammary gland decreases and is replaced with fat as people age, hence the density of the mammary gland can be estimated from the age of the examinee. The ratio of fat can be expressed by BMI.

In step S902, classification of the attributes is determined. If the attribute is age, then three types of classification criteria (20s or younger, 30s, 40s or older, for example, can be used. If the attribute is BMI, then three types of classification criteria (0 to 20, 20 to 30, 30 or more), for example, can be used. Each step of S901 and S902 may be implemented by the user performing each setting in the information processing apparatus using the input device.

In step S903, the information processing apparatus 500 determines the ratio of the components constituting the breast (object) based on the inputted information. In other words, the information processing apparatus 500 acquires the component ratio referring to the database (not illustrated) based on the parameters and the attribute classification.

In step S904, the information processing apparatus 500 determines the effective attenuation coefficient μ_{eff} for each classification criteria of the component. Then in step S905, the oxygen saturation considering the light quantity distribution of the breast in the depth direction is calculated, using the effective attenuation coefficient. In other words, the ratio of the components and the effective attenuation coefficient of each component are known, hence the effective attenuation coefficient of the entire object can be calculated with weighting based on each ratio. Then the irradiation light quantity on the surface of the object is determined based on the assumed light quantity when the light has been emitted, and the Monte Carlo method or the like is performed using this effective attenuation coefficient, whereby the light quantity at any position inside the object can be calculated. A plurality of light quantity values may be assumed, so that calculation is performed for each light quantity value.

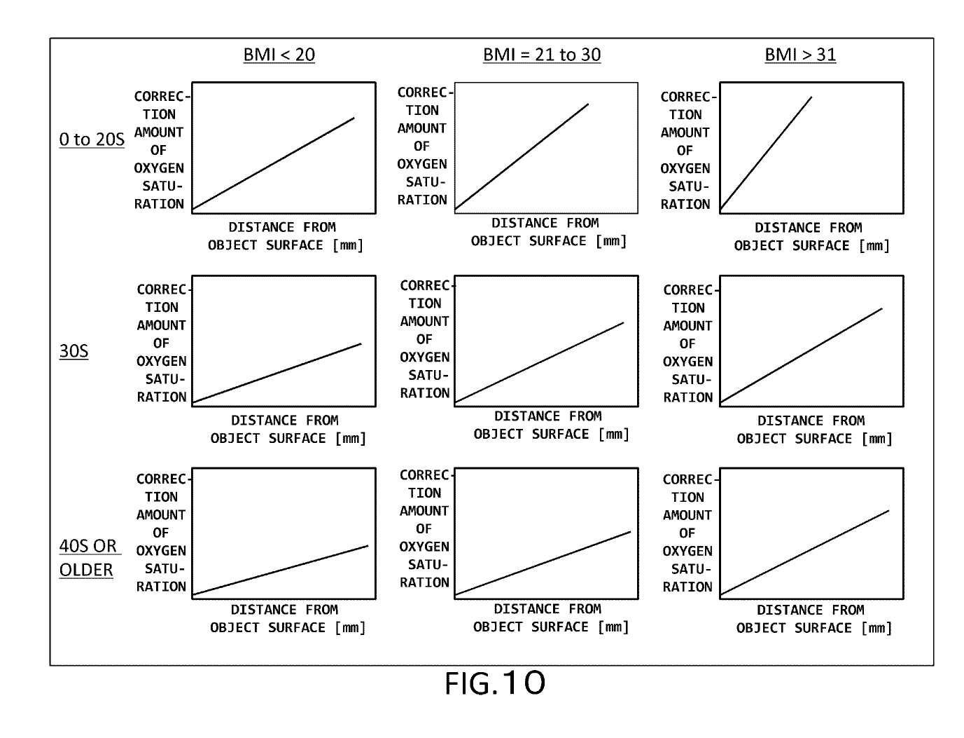

In step S906, the simplified oxygen saturation is calculated assuming that the light quantity distribution does not exist in the depth direction, and the light quantity distribution inside the object is uniform. In step S907, the amount of deviation of the oxygen saturation is calculated for all the classifications, and the correction table thereof is created and stored. FIG. 10 is an example of the correction table, which expresses the amount of deviation of the oxygen saturation, which is the correction amount, for each classification. The information on the correction may be created not as a correction table, but as a correction formulae.

The effective attenuation coefficient μ_{eff} can also be measured by the time resolved spectroscopy (TRS). The effective attenuation coefficient μ_{eff} of the breast of the examinee, which matches each classification, may actually be measured by the time resolved spectroscopy, and the light quantity distribution may be calculated using the actually measured effective attenuation coefficient.

b: Oxygen Saturation Degree Deriving Step for Each Examinee

In step (b), using the correction table created in step (a), the correction processing is performed for the oxygen saturation distribution data based on the detection signal acquired by actual photoacoustic measurement. In step S908, for the detection signal for each wavelength acquired by the photoacoustic measurement, the information processing apparatus 500 performs: the initial sound pressure distribution acquiring processing; the simplified absorption coefficient distribution acquiring processing in which the light quantity distribution ϕ (x, y, 0) on the surface of the breast is applied to all the depths; and the simplified oxygen saturation acquiring processing, just like Embodiment 1. In S908, the detection signal acquired in the photoacoustic measurement, which has been actually performed immediately before S908, may be used, or the detection signal acquired in the photoacoustic measurement, which has been performed at a timing different from step (b), may be read from the storage device (not illustrated) and used.

In step S909, the information processing apparatus 500 determines the examinee attributes based on the age of the examinee and the BMI. The examinee attributes may be determined based on the input by the user via the input device. If the information on the examinee has already been registered in the storage device of the information processing apparatus, the user may input the information which identifies this examinee.

In step S910, the correction amount of the oxygen saturation is selected from the correction table in FIG. 10. In step S911, the selected correction amount is subtracted from the simplified oxygen saturation which has been determined in S908, whereby the correction process is executed.

Embodiment 3 as well implements the advantage of the present invention, that is, a highly accurate oxygen saturation can be acquired without accurately calculating the light quantity distribution. Further, a desired correction table can be referred to for each attribute of the examinee.

In the above mentioned flow, the correction table is created by the calculation based on the attributes of the examinee, but the present invention is not limited to this method. For example, when Embodiment 1 is applied to a sufficient number of examinees, the correction data can be acquired for each examinee. This correction data may be classified by examinee attributes, whereby the correction table is created. In this case, the correction data may be simply averaged for each classification of the examinee attribute, or may be expressed as a function. The correction amount which has been applied to an examinee having equivalent attributes in the past may be referred to.

If the oxygen saturation of the examinee was acquired in the past using the method according to Embodiment 1 or 2, correction may be performed with reference to the correction amount determined at that time.

According to the method of Embodiment 3, correction is performed for the entire object, hence there is no need to extract blood vessels in step (b).

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-018688, filed on Feb. 3, 2017, which is hereby incorporated by reference herein in its entirety.

What is claimed is:

1. An information processing apparatus, comprising:
an information processing unit configured to:
acquire concentration information, which indicates a spatial distribution of concentration of a substance constituting an object and which originates from a photoacoustic wave generated by light irradiation of the object; and
correct, based on the concentration in a specific position of the object in the concentration information, the concentration in a deep region of the object deeper than the specific position in the concentration information.

2. The information processing apparatus according to claim 1, wherein the specific position is included in a region near a surface of the object.

3. The information processing apparatus according to claim 2, wherein
the information processing unit is configured to:
acquire oxygen saturation information as the concentration information based on a photoacoustic wave generated by an irradiation of the object with first light having a first wavelength and a photoacoustic wave generated by an irradiation of the object with second light having a second wavelength different from the first wavelength, and
acquire a characteristic information based on at least one of the photoacoustic wave corresponding to the first light and the photoacoustic wave corresponding to the second light,
extract a first blood vessel based on the characteristic information,
correct an oxygen saturation of the first blood vessel in the deep region based on an oxygen saturation of the first blood vessel in the region near the surface of the object.

4. The information processing apparatus according to claim 3, wherein the information processing unit is configured to:
correct an oxygen saturation of the entire first blood vessel based on the oxygen saturation of the first blood vessel in the region near the surface of the object.

5. The information processing apparatus according to claim 3, wherein the information processing unit is configured to:
extract a second vessel which is not positions in the region near the surface of the object based on the characteristic information, and
correct an oxygen saturation of the second vessel based on the oxygen saturation of the first blood vessel in the region near the surface of the object.

6. The information processing apparatus according to claim 5, wherein
the information processing unit is configured to correct the oxygen saturation of the second blood vessel based on first relationship information which indicates a relationship between the oxygen saturation and a distance from the surface of the object for the first blood vessel, and second relationship information which indicates the relationship between the oxygen saturation and a distance from the surface of the object for the second blood vessel.

7. The information processing apparatus according to claim 3, wherein
the characteristic information includes an initial sound pressure distribution or an absorption coefficient distribution.

8. The information processing apparatus according to claim 1, wherein
the information processing unit is configured to:
acquire correction information for correcting the concentration information based on attributes of the object, and
correct the concentration information based on the concentration in the specific position of the object and the correction information.

9. The information processing apparatus according to claim 8, wherein
the correction information is a correction table or a correction formulae for the concentration information, the correction table or correction formulae being stored for each attribute of the object.

10. The information processing apparatus according to claim 8, wherein
the attributes include at least any one of age, height, weight, BMI, breast size, race and medical history.

11. The information processing apparatus according to claim 1, wherein
the deep region is a position of which distance from the surface of the object on the normal line that is on the surface and intersects the specific position is longer than that of the specific position.

12. An information processing method, comprising:
an information processing step of acquiring concentration information, which indicates a spatial distribution of concentration of a substance constituting an object, and which originates from a photoacoustic wave generated by light irradiation of the object; and
a correction step of, based on the concentration in a specific position of the object in the concentration information, correcting the concentration in a deep region of the object deeper than the specific position of the object in the concentration information.
